(19) United States
(12) Patent Application Publication
(10) Pub. No.: US 2015/0122252 A1
(43) Pub. Date: May 7, 2015

(54) HAND-HELD PERSONAL VAPORIZER

(71) Applicant: Kevin Frija, Dania Beach, FL (US)

(72) Inventor: Kevin Frija, Dania Beach, FL (US)

(21) Appl. No.: 14/153,009

(22) Filed: Jan. 11, 2014

Related U.S. Application Data
(60) Provisional application No. 61/898,666, filed on Nov. 1, 2013.

(51) Int. Cl. A24F 47/00; A61M 15/00; A61B 5/00; A61M 15/06; A61B 5/117

(52) U.S. Cl. CPC A24F 47/008; A61M 15/06; A61B 5/1172; A61B 5/742; A61B 5/0022; A61M 15/002
USPC 128/202.21

(57) ABSTRACT

A hand-held personal vaporizer including a cylindrical housing and a vaporizer module disposed within the cylindrical housing. The vaporizer includes a vaporizer control module functionally coupled to the vaporizer and configured to control function of the vaporizer. The vaporizer control module automatically restrict use of the vaporizer after a predefined script is executed. The vaporizer includes a fingerprint scanner functionally coupled to a push button and configured to scan a fingerprint. The hand-held personal vaporizer includes a locking module in communication with the vaporizer control module, and functionally coupled to the fingerprint scanner. The locking module is configured to securely lock the vaporizer from use if an authorized fingerprint is not detected through the fingerprint scanner.

### CROSS-REFERENCE TO RELATED APPLICATIONS

This invention claims priority, under 35 U.S.C. §120, to the U.S. Provisional Patent Application No. 61/898,666 to Kevin Frija filed on Nov. 1, 2013, which is incorporated by reference herein in its entirety.

### BACKGROUND OF THE INVENTION

1. Field of the Invention

The present invention relates to vaporizers, specifically to a hand-held personal vaporizer.

2. Description of the Related Art

Vaporization is an alternative to burning (smoking) that avoids the inhalation of many irritating toxic and carcinogenic by-products. Vaporizers contain various forms of extraction chambers including straight bore, venturi, or sequential venturi, and are made of materials such as metal or glass. The extracted vapor may be collected in a jar or inflatable bag, or inhaled directly through a hose or pipe. Some vaporizers include devices and/or structures for causing fluids to vaporize, such as but not limited to ultrasonic vaporization devices that use ultrasound to convert fluids to vapors.

With little to no smoke produced and generally cooler temperatures than what occurs when material is burned, less material is required to achieve a given level of effect. Hence, the irritating and harmful effects of smoking are reduced, as is secondhand smoke.

E-cigarette or Electronic Cigarette is generally a subcategory of vaporizers designed to replace cigarettes. E-cigarettes are handheld personal vaporizers that make use of E-Liquid: flavoring and nicotine generally dissolved in a solution of vegetable glycerine and/or propylene glycol. Using E-cigarettes, as opposed to lit cigarettes, is considered to overall be a safer experience for both users and those nearby.

Because of the benefits of vaporization over smoking, e-cigarettes are often regarded among the medical community as both as a valuable harm reducing alternative to lit cigarettes and as an effective smoking cessation aid. Vapor preferred by many users is higher in concentration and aromatic density due to the increased rate of extraction and smaller sample size.

Some improvements have been made in the field. Examples of references related to the present invention are described below in their own words, and the supporting teachings of each reference are incorporated by reference herein:

U.S. Patent Application Publication No.: 2013/0233309, by Todd, discloses a new technology that enables administration of a vaporizable substance to patients for medical purposes. In one embodiment, this invention is a component of a system of technologies, processes and concepts that creates a new method of producing, delivering, administering and regulating the use of medical vaporizable substances.

U.S. Pat. No. 7,806,938, issued to Ng et al., discloses a method and apparatus are described that control the supply of electrical power to one or more components of a computing device. Other embodiments are described and claimed.

U.S. Pat. No. 7,525,417, issued to Strader et al., discloses a door lock is provided with a biometric sensor and a key cylinder. The biometric sensor is protected by being positioned on a shield, and such that the sensor is not exposed to damage by being on the front face of the door lock. The shield extends vertically downwardly from the remainder of the front face of the door lock, and the sensor is mounted to an inner face of the shield. Thus, the sensor is spaced away from a door when the door lock is mounted on a door. A finger may be inserted into a space between the sensor and the door, and the sensor can then capture information about the fingerprint. This information can be compared to expected fingerprints, and entry into a building can be granted based upon a recognition of this fingerprint.

U.S. Pat. No. 7,844,363, issued to Mehdizadeh, discloses enabling an individual to conveniently purchase herbal medications and prescription medicines from specialized vending machines. The system provides for the individual to be processed through a central database to be certain that the item being purchased has been legally authorized by an appropriate medical authority such as a licensed physician and has provided appropriate verification to confirm that the individual who is receiving the medication is the correct individual. The present invention enables the individual to conveniently purchase the medication from a vending machine.

U.S. Patent Application Publication No.: 2013/0231046, by Pope et al., discloses An electronic device may have electrical components such as sensors. A sensor may have sensor circuitry that gathers sensor data using a conductive structure. The sensor may be a touch sensor that uses the conductive structure to form a capacitive touch sensor electrode or may be a fingerprint sensor that uses the conductive structure with a fingerprint electrode array to handle fingerprint sensor signals. Near field communications circuitry may be included in an electronic device. When operated in a sensor mode, the sensor circuitry may use the conductive structure to gather a fingerprint or other sensor data. When operated in near field communications mode, the near field communications circuitry can use the conductive structure to transmit and receive capacitively coupled or inductively coupled near field communications signals. A fingerprint sensor may have optical structures that communicate with external equipment.

The inventions heretofore known suffer from a number of disadvantages which include being limited in use, being limited in security, being limited in dosage control, being limited in safety, failing to lock out unauthorized users, failing to manage multiple user accounts, being bulky, not coordinating with third parties, not being safe, and failing to authenticate a user before operation.

What is needed is a hand-held personal vaporizer that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available vaporizers. Accordingly, the present invention has been developed to provide an effective and efficient hand-held personal vaporizer for safe inhalation of vapor.

According to one embodiment of the invention, there is a hand-held personal vaporizer. The vaporizer may include a cylindrical housing that may have a mouthpiece and an elongated region opposite the mouthpiece. The hand-held personal vaporizer may include a vaporizer module disposed within the cylindrical housing. The hand held-personal vaporizer may include a vaporizer control module that may be functionally coupled to the vaporizer and may be configured to control function of the vaporizer. The vaporizer control module may automatically restrict use of the vaporizer after a predefined script is executed.

The hand-held personal vaporizer may include a display on the cylindrical housing and may be functionally coupled to the vaporizer control module. The vaporizer may include a push button that may be disposed on the elongated region of the cylindrical housing, and may be functionally coupled to the vaporizer control module. The vaporizer may include a fingerprint scanner that may be functionally coupled to the push button and may be configured to scan a fingerprint. The fingerprint scanner may be disposed on a flat platform on a top surface of the cylindrical housing, under which there may be a button mechanism.

The hand-held personal vaporizer may include a locking module that may be in communication with the vaporizer control module, and may be functionally coupled to the fingerprint scanner. The locking module may be configured to securely lock the vaporizer from use if an authorized fingerprint is not detected through the fingerprint scanner.

The vaporizer may include a preference module that may be in communication with the vaporizer control module, and may be configured to store preference settings for a stored user account. The vaporizer may include a dosage settings module that may be functionally coupled to the vaporizer control module and may be configured to store dosage settings per user. The hand-held personal vaporizer may include a data storage module that may be in communication with the vaporizer control module, and may be configured to store user data, such as dosage and preferences from the vaporizer.

The vaporizer may include a second fingerprint scanner that may be functionally coupled to the vaporizer control module and may be located remote from the vaporizer on a personal computing device. The vaporizer control module may only allow operation of the vaporizer when authorized fingerprints are detected at both the first and second fingerprint scanners. The vaporizer may include a communication module that may be in communication with the vaporizer control module, and may be configured to provide wireless communication to the modules and components of the vaporizer.

The hand-held personal vaporizer may include a remote locking module that may be functionally coupled to the vaporizer control module and may be located remote from the vaporizer on a personal computing device and wherein the vaporizer control module only allows operation of the vaporizer when an authorized fingerprint is detected at fingerprint scanners and the remote locking module authorizes the use.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a perspective view of a hand-held personal vaporizer, according to one embodiment of the invention;

FIG. 2 is a top plan view of a hand-held personal vaporizer, according to one embodiment of the invention;

FIG. 3 is a top plan view and a perspective view of a hand-held personal vaporizer, according to one embodiment of the invention;

FIG. 4 is a module diagram of a hand-held personal vaporizer, according to one embodiment of the invention; and

FIG. 5 is a network diagram illustrating a hand-held personal vaporizer in communication with a third-party control module over a network.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Many of the functional units described in this specification have been labeled as modules in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like. Modules may also be implemented in software for execution by various types of processors. An identified module of programmable or executable code may, for instance, comprise one or more physical or logical blocks of computer instructions which may, for instance, be organized as an object, procedure, or function.

Nevertheless, the executables of an identified module need not be physically located together, but may comprise disparate instructions stored in different locations which, when joined logically together, comprise the module and achieve the stated purpose for the module. Indeed, a module and/or a program of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

The various system components and/or modules discussed herein may include one or more of the following: a host server, motherboard, network, chipset or other computing system including a processor for processing digital data; a memory device coupled to a processor for storing digital data; an input digitizer coupled to a processor for inputting digital data; an application program stored in a memory device and accessible by a processor for directing processing of digital data by the processor; a display device coupled to a processor and/or a memory device for displaying information derived from digital data processed by the processor; and a plurality of databases including memory device(s) and/or hardware/software driven logical data storage structure(s).

Various databases/memory devices described herein may include records associated with one or more functions, purposes, intended beneficiaries, benefits and the like of one or more modules as described herein or as one of ordinary skill in the art would recognize as appropriate and/or like data useful in the operation of the present invention.

As those skilled in the art will appreciate, any computers discussed herein may include an operating system, such as but not limited to: Andriod, iOS, BSD, IBM z/OS, Windows Phone, Windows CE, Palm OS, Windows Vista, NT, 95/98/2000, OS X, OS2; QNX, UNIX; GNU/Linux; Solaris; MacOS; and etc., as well as various conventional support software and drivers typically associated with computers. The computers may be in a home, industrial or business environment with access to a network. In an exemplary embodiment, access is through the Internet through a commercially-available web-browser software package, including but not limited to Internet Explorer, Google Chrome, Firefox, Opera, and Safari.

The present invention may be described herein in terms of functional block components, functions, options, screen shots, user interactions, optional selections, various processing steps, features, user interfaces, and the like. Each of such described herein may be one or more modules in exemplary embodiments of the invention even if not expressly named herein as being a module. It should be appreciated that such functional blocks and etc. may be realized by any number of hardware and/or software components configured to perform the specified functions. For example, the present invention may employ various integrated circuit components, e.g., memory elements, processing elements, logic elements, scripts, look-up tables, and the like, which may carry out a variety of functions under the control of one or more microprocessors or other control devices. Similarly, the software elements of the present invention may be implemented with any programming or scripting language such as but not limited to Eiffel, Haskell, C, C++, Java, Python, COBOL, Ruby, assembler, Groovy, PERL, Ada, Visual Basic, SQL Stored Procedures, AJAX, Bean Shell, and extensible markup language (XML), with the various algorithms being implemented with any combination of data structures, objects, processes, routines or other programming elements. Further, it should be noted that the present invention may employ any number of conventional techniques for data transmission, signaling, data processing, network control, and the like. Still further, the invention may detect or prevent security issues with a client-side scripting language, such as JavaScript, VBScript or the like.

Additionally, many of the functional units and/or modules herein are described as being “in communication” with other functional units, third party devices/systems and/or modules. Being “in communication” refers to any manner and/or way in which functional units and/or modules, such as, but not limited to, computers, networks, mobile devices, program blocks, chips, scripts, drivers, instruction sets, databases and other types of hardware and/or software, may be in communication with each other. Some non-limiting examples include communicating, sending, and/or receiving data and metadata via: a wired network, a wireless network, shared access databases, circuitry, phone lines, internet backbones, transponders, network cards, busses, satellite signals, electric signals, electrical and magnetic fields and/or pulses, and/or so forth.

As used herein, the term “network” includes any electronic communications means which incorporates both hardware and software components of such. Communication among the parties in accordance with the present invention may be accomplished through any suitable communication channels, such as, for example, a telephone network, an extranet, an intranet, Internet, point of interaction device (point of sale device, personal digital assistant, cellular phone, kiosk, etc.), online communications, off-line communications, wireless communications, transponder communications, local area network (LAN), wide area network (WAN), networked or linked devices and/or the like. Moreover, although the invention may be implemented with TCP/IP communications protocols, the invention may also be implemented using other protocols, including but not limited to IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997), the contents of which are hereby incorporated by reference.

Reference throughout this specification to an “embodiment,” “e.g.” an “example” or similar language means that a particular feature, structure, characteristic, or combinations thereof described in connection with the embodiment is included in at least one embodiment of the present invention and is non-limiting. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of and” consisting essentially of.”

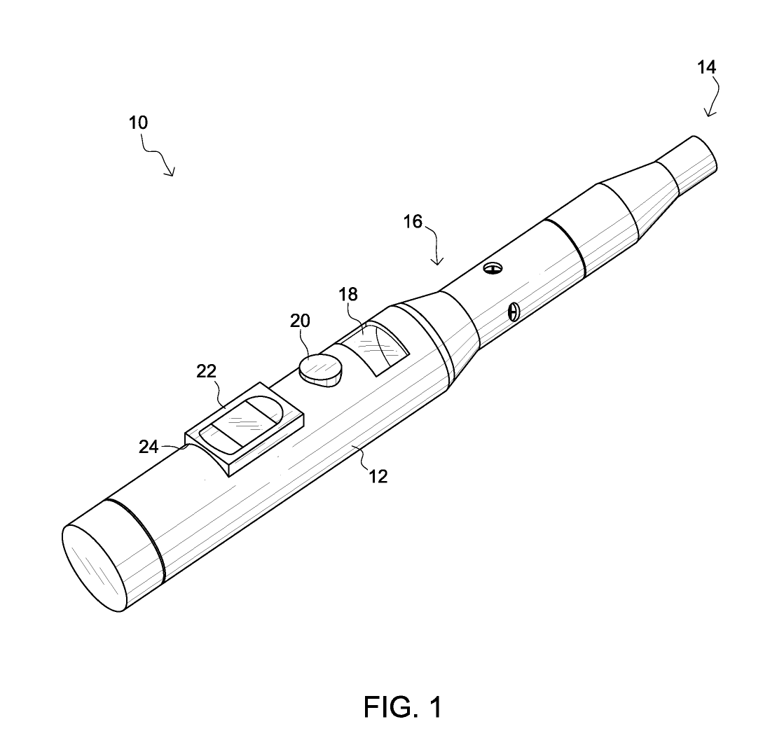

FIG. 1 is a perspective view of a hand-held personal vaporizer, according to one embodiment of the invention. There is shown a hand-held (i.e. carried and operable with a single hand when in use) personal (i.e. providing vapor to a single person at a time) vaporizer 10 including a cylindrical housing 12 having a mouthpiece 14.

The illustrated hand held vaporizer 10 includes a cylindrical housing 12 having a mouthpiece 14 and an elongated region 16 opposite the mouthpiece 14. The hand-held personal vaporizer 10 includes a display 18 on the cylindrical housing 12 and functionally coupled to a vaporizer control module. The vaporizer 10 includes a push button 20 disposed on the elongated region 16 of the cylindrical housing 12, functionally coupled to the vaporizer control module. The vaporizer 10 includes a fingerprint scanner 22 functionally coupled to the vaporizer control module and configured to scan a fingerprint, wherein the control module restricts or otherwise modifies operation of the vaporizer unless a valid fingerprint is scanned by the fingerprint scanner. Such a system may not operate at all (except for scanning fingerprints), unless and until a valid fingerprint stored in memory of the device is scanned.

The illustrated fingerprint scanner 22 is disposed on a flat platform on a top surface 24 of the cylindrical housing 12. The flat platform provides a larger surface for the fingerprint scanner than would otherwise be provided by the cylindrical housing without substantially increasing the size of the cylindrical housing and without interfering with the aesthetics of the cylindrical design. The flat platform is raised (extends outwardly from the cylindrical housing) and also provides a mounting region on which the fingerprint scanner may be mounted. The flat platform also provides lateral support and protection for the fingerprint scanner, which is nested inside the flat platform, the edges of the flat platform being walls of protection for the same. Advantageously, the flat platform also provides an easy non-visual cue as to where one would place ones finger when needed to be scanned.

Advantageously, the hand-held personal vaporizer may be sized very small, similar to the size of a cigarette, while still permitting biometric control, even though cigarettes are generally smaller than fingers and the effective flat region of a cylindrical housing sized similar to a cigarette is much smaller than the width of a typical fingerprint scanner.

According to one embodiment of the invention, there is a finger print scan module and/or mechanism disposed on a hand-held personal vaporizer (e.g. electronic cigarette device) to allow a locking or unlocking of the vaporizer, by identifying and/or authenticating the user, as well as other features, functions, and or information being user identified.

According to one embodiment of the invention, there may be a button with finger print scan built in and/or small display screen able to scan finger print combined with personal vaporizer and or e-cigarette. There may be a button with a display screen and a fingerprint scanner all built into one, such that the button may display information thereon, may scan a finger pressed onto the button and may activate when pressed (as a button). The owner/user of the device may lock out unauthorized users and also add other users as needed and each user may have personal preferences and or dosage settings as well as information collect about their use and shared with other programs or application to monitor use by themselves, professionals (e.g. doctors, therapists, healers), supervisory authorities (e.g. the government, parents, guardians), or others.

According to one embodiment of the invention, there is a button/screen/other finger print capture method or other personal identifying scan i.e.: biometric scans, such as but not limited to retinal scanning. The location of one or more such scanning mechanisms may be in/on the vaporizer and/or on/in a smartphone/tablet/etc. paired to vaporizer (in communication with the vaporizer), e.g. an electronic application on a smartphone that allows the vaporizer to function using information from a third-party scanner. The third-party scanner may have master control over operation of the personal hand-held vaporizer such that even if an authenticated fingerprint scan occurs at the vaporizer, the vaporizer will not function without authorization from the third-party scanner.

There may be a vapor device having a transponder in functional communication with an on/off module (that controls operation of the vapor device) in the unit such that the on/off module may be activated by the transponder on receipt of a particular the transponder being activated by a remote device, including but not limited to a hand held ‘fob’ that you carry as a ‘key’ to turn on the device.

There may be a vapor device having a Bluetooth brand or other near-field communication system in communication with an on/off module that may be activated remotely, such as but not limited to either being triggered by a finger print scan from your smart phone or direction or just from an application in the phone to turn on or off where phone, tablet, app, etc. functions as a key.

There may be a vapor device including a module to transmit information back to phone application or any computer on usage information, e.g. how many puffs or doses of medicine or who the user is and where he is and/or location back to phone application.

In one non-limiting embodiment, there is a hand-held personal vaporizer having a unique identifier (e.g. serial code) and a communication module that permits it to access a network. There is also a smartphone/tablet application that a user may download that includes account management software/tools/modules and allows them to create an account that is linked to the unique identifier of the hand-held personal vaporizer. The application may include a fingerprint scanning module that uses the camera or an attached/coupled peripheral device to do a biometric scan (generally a fingerprint scan) of the user and associate that with the unique vaporizer through the account. The account data may be synchronized with the vaporizer, wherein at least some data associated with the account is stored in the vaporizer. Other account details may be selected, such as but not limited to dosage limits, allowed materials to be used with the vaporizer, characteristics of the user, goals, dosage requirements, prescription details, and the like and such information may be also saved in memory in the device. The display of the device may display such information and/or may automatically display information related thereto, such as but not limited display changing color to that a dosing is overdue for the user of the device. Use of the vaporizer may require one or more fingerprint or other biometric scans through the vaporizer itself and/or through the application. The application may be operated, initialized, and/or managed by a third party (parent, doctor, caregiver, etc.) or by the user. The application may be linked to a plurality of unique devices and/or may be associated with a plurality of accounts.

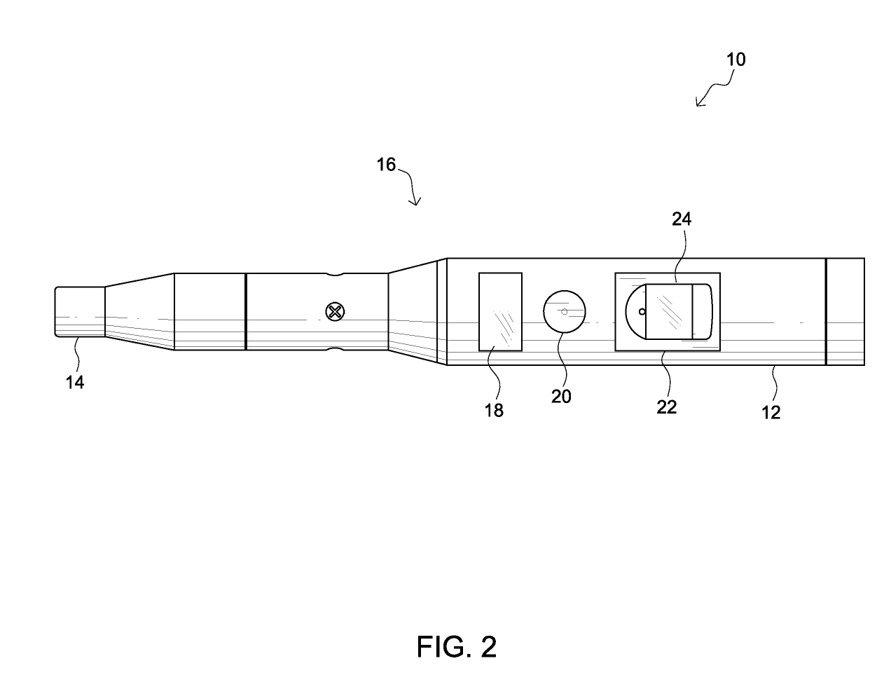

FIG. 2 is a top plan view of a hand-held personal vaporizer, according to one embodiment of the invention. There is shown a hand-held personal vaporizer 10 including a cylindrical housing 12 having a mouthpiece 14, a display 18, and a fingerprint scanner 22.

The illustrated hand-held personal vaporizer 10 includes a cylindrical housing 12 having a mouthpiece 14 and an elongated region 16 opposite the mouthpiece 14. The mouthpiece 14 includes a hole through which vapor is dispensed (or is drawn when the mouthpiece is sucked on by the user) and an exterior region on which the user's lips find purchase during use.

The illustrated hand-held personal vaporizer 10 includes a vaporizer module disposed within the cylindrical housing 12. The vaporizer module includes such hardware needed to cause vaporization of fluid and/or other materials disposed within the housing that are intended to be vaporized and consumed by the user. Such a module may include one or more of a heating element, an ultrasonic vapor generator (vaporizer, such as those found in humidifiers and the like), forced air vaporizers and the like and combinations thereof. Such are generally controlled by controlling the current flowing through the device.

The illustrated hand held-personal vaporizer 10 includes a vaporizer control module functionally coupled to the vaporizer and configured to control function of the vaporizer 10. The vaporizer control module automatically restrict use of the vaporizer 10 after a predefined script is executed, such as but not limited to after registering a predefined number of puffs, a predefined period of time, a predefined volume of fluid being used, and/or combinations thereof.

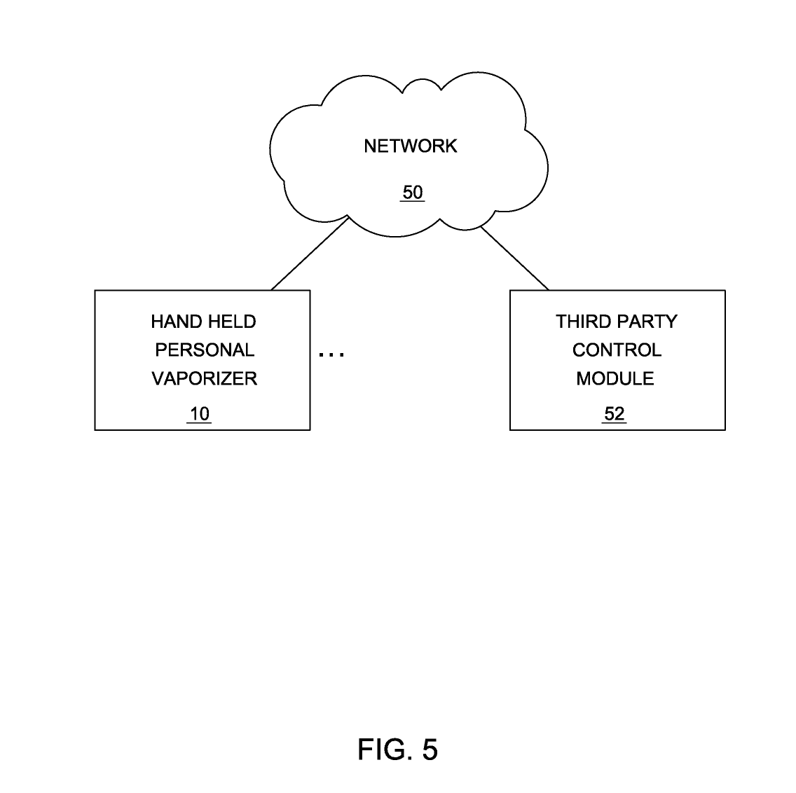

The illustrated hand-held personal vaporizer 10 includes a display 18 on the cylindrical housing 12 and functionally coupled to the vaporizer control module. The display may be functionally coupled to the control module such that information therefrom may be displayed on the display. The display may simply be a single LED light or may be more complicated, such as but not limited to: a multi-colored LED light (wherein different colors mean different things), a bank or array of LED lights, a 2D LED display, electronic paper and the like and combinations thereof. The display may display account information from the control module (or a third-party control module, See FIG. 5). The display may prompt the user for actions that are required, e.g. Scan Finger (before allowing the button to function).

The vaporizer 10 includes a push button 20 disposed on the elongated region 16 of the cylindrical housing 12, is functionally coupled to the vaporizer control module. The vaporizer 10 includes a fingerprint scanner 22 functionally coupled to the control module and configured to scan a fingerprint. The fingerprint scanner 22 is disposed on a flat platform on a top surface 24 of the cylindrical housing 12.

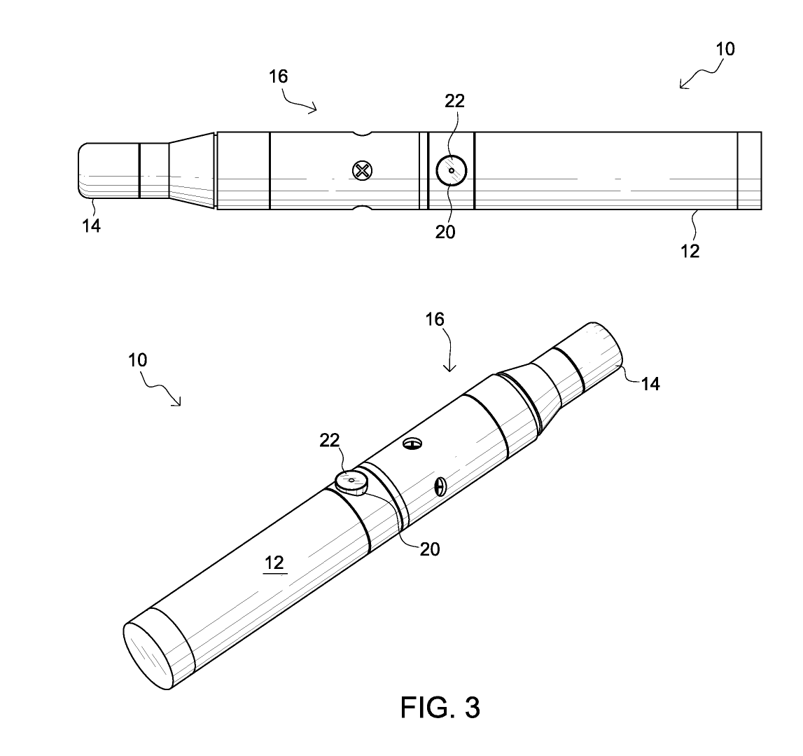

FIG. 3 is a top plan view and a perspective view of a hand-held personal vaporizer, according to one embodiment of the invention. There is shown a hand-held personal vaporizer 10 including a cylindrical housing 12 having a mouthpiece 14, a push button 20 having a fingerprint scanner 22.

The illustrated hand-held personal vaporizer includes a cylindrical housing 12 having a mouthpiece 14 and an elongated region 16 opposite the mouthpiece 14. The hand-held personal vaporizer 10 includes a vaporizer module disposed within the cylindrical housing 12. The hand held-personal vaporizer 10 includes a vaporizer control module functionally coupled to the vaporizer and configured to control function of the vaporizer.

The hand-held personal vaporizer 10 includes a display 18 on the cylindrical housing 12. The display 18 is functionally coupled to the vaporizer control module. The vaporizer 10 includes a push button 20 disposed on the elongated region 16 of the cylindrical housing 12. The push button is functionally coupled to the vaporizer control module. The illustrated vaporizer 10 includes a fingerprint scanner 22 functionally disposed on the push button 20 and configured to scan a fingerprint. As illustrated, the push button 20 and the fingerprint scanner 22 are combined in one push button mechanism disposed on the cylindrical housing, such that pushing the button with your finger also permits the fingerprint scanner to scan that same finger at the region where the finger touches the button. In particular, the button mechanism that allows for the button to be depressed and activates on depression is disposed at the base of the fingerprint scanner, thereby providing multifunction with a smaller space requirement. In such an embodiment, there may be a spring or bias member disposed below a platform on which rests the fingerprint scanner, wherein the fingerprint scanner is coupled to the cylindrical housing by a belt of wires through which information from the scanner and power to the scanner may flow. Below the platform is a switch mechanism whereby if the platform is depressed against the force of the spring sufficiently to touch the switch mechanism, the switch mechanism activates. Accordingly, the person may push on the fingerprint scanner, which may activate the scan and/or activate the device itself, though activation of the vaporizer may be delayed until the control module verifies the scanned fingerprint.

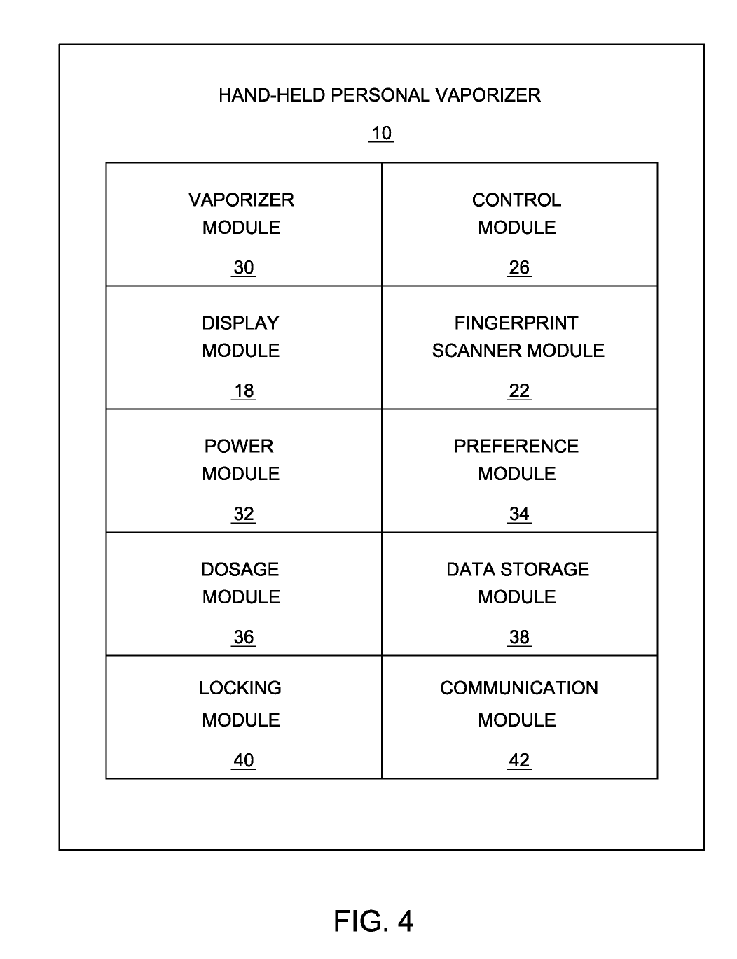

FIG. 4 is a module diagram of a hand-held personal vaporizer, according to one embodiment of the invention. There is shown a hand-held personal vaporizer 10 including a vaporizer module 30, a control module 26, a display module 18, a fingerprint scanner module 22, a power module 32, a preference module 34, a dosage module 36, a data storage module 38, a locking module 40, and a communication module 42.

The illustrated hand-held personal vaporizer 10 includes a vaporizer module 30 disposed within a cylindrical housing. The vaporizer module 30 is configured to vaporizer liquid into vapor for inhalation. Non-limiting examples of a vaporizer module may be a vaporizer module as described in WO2008145177; or a vaporization system as described in U.S. Pat. No. 5,835,678, which are incorporated for their supporting teachings herein.

The hand held-personal vaporizer 10 includes a vaporizer control module 26 functionally coupled to the vaporizer 10 and configured to control function of the vaporizer 10 and the modules and components thereof. The vaporizer control module 26 automatically restricts use of the vaporizer after a predefined script is executed. Non-limiting examples of a vaporizer control module may be a control module described in U.S. Pat. No. 5,430,836, issued to Wolf et al.; or a control module described in U.S. Pat. No. 6,243,635, issued to Swan et al. which are incorporated for their supporting teachings herein. A control module may include but is not limited to a processor, a state machine, a script, a decision tree, and the like.

The hand-held personal vaporizer 10 includes a display module 18 disposed on a cylindrical housing and functionally coupled to the vaporizer control module 26. The display module is configured to display settings, preferences, dosage, etc. data of the vaporizer and usage thereof. Non-limiting examples of a display module may be a display/interface module as described in U.S. Pat. No. 6,272,562, issued to Scott et al.; a touch screen interface module as described in U.S. Pat. No. 5,884,202 and U.S. Pat. No. 6,094,609, issued to Arjomand, which are incorporated for their supporting teachings herein.

The vaporizer 10 includes a push button disposed on an elongated region of the cylindrical housing, and is functionally coupled to the vaporizer control module 26. The vaporizer 10 includes a fingerprint scanner 22 functionally coupled to the push button 20 and configured to scan a fingerprint. The fingerprint scanner 22 is disposed on a flat platform on a top surface of the cylindrical housing, under which there may be a button mechanism. Non-limiting examples of a fingerprint scanner may be a fingerprint scanner as described in U.S. Pat. No. 8,111,136; or a fingerprint scanning device as described in U.S. Pat. No. 7,391,006, which are incorporated for their supported teachings herein.

The hand-held personal vaporizer 10 includes a locking module 40 in communication with the vaporizer control module 26, and functionally coupled to the fingerprint scanner 22. The locking module 40 is configured to securely lock the vaporizer 10 from use if an authorized fingerprint is not detected through the fingerprint scanner 22. Non-limiting examples of a locking module may be a locking module as described in U.S. Pat. No. 8,270,173; or a locking module as described in U.S. Patent Publication No.: 2012/0212887, which are incorporated for their supported teachings herein.

The vaporizer 10 include a preference module 34 in communication with the vaporizer control module 26, and configured to store preference settings for a stored user account. Non-limiting examples of a preference module may be a preference module as described in U.S. Pat. No. 8,351,763; or a profile module as described in WO2013130834, which are incorporated for their supported teachings herein.

The vaporizer 10 includes a dosage settings module 36 functionally coupled to the vaporizer control module 26 and configured to store dosage settings per user. Non-limiting examples of a dosage settings module may be a does control module as described in U.S. Pat. No. 7,150,741; or a does limiter module as described in WO2013068483, which are incorporated for their supported teachings herein.

The hand-held personal vaporizer 10 include a data storage module 38 in communication with the vaporizer control module 26, and configured to store user data, such as dosage and preferences from the vaporizer 10. Data storage modules may be databases or data files, and the memory storage device may be hard drives or tapes. A non-limiting example of a data base is Filemaker Pro 11, manufactured by Filemaker Inc., 5261 Patrick Henry Dr., Santa Clara, Calif., 95054. Non-limiting examples of a storage module may include: a HP Storage Works P2000 G3 Modular Smart Array System, manufactured by Hewlett-Packard Company, 3000 Hanover Street, Palo Alto, Calif., 94304, USA; or a Sony Pocket Bit USB Flash Drive, manufactured by Sony Corporation of America, 550 Madison Avenue, New York, N.Y., 10022.

The vaporizer 10 includes a second fingerprint scanner 50 functionally coupled to the vaporizer control module 26 and located remote from the vaporizer 10 on a personal computing device. The vaporizer control module 26 only allows operation of the vaporizer 10 when authorized fingerprints are detected at both the first and second fingerprint scanners.

The vaporizer 10 includes a communication module 42 in communication with the vaporizer control module 26, and configured to provide wireless communication to the modules and components of the vaporizer 10. Non-limiting examples of a communication module may be but not limited to: a communication module described in U.S. Pat. No. 5,307,463, issued to Hyatt et al.; or a communication module described in U.S. Pat. No. 6,133,886, issued to Fariello et al. which are incorporated for their supporting teachings herein.

The hand-held personal vaporizer 10 includes a remote locking module functionally coupled to the vaporizer control module 26 and located remote from the vaporizer 10 on a personal computing device and wherein the vaporizer control module 26 only allows operation of the vaporizer 10 when an authorized fingerprint is detected at fingerprint scanners and the remote locking module authorizes the use. The remote locking module may be in communication with the locking module 40 of the vaporizer and may be configured to provider operational controls thereto.

FIG. 5 is a network diagram illustrating a hand-held personal vaporizer in communication with a third-party control module over a network. There is shown a hand-held personal vaporizer 10 (which may represent a plurality of such devices connected in various manners to the network) in communication with a network 50 to which a third-party control module 52 is also connected. Accordingly, the third-party control module and the hand-held personal vaporizer may communicate and provide information and/or instructions one to the other.

The illustrated hand-held personal vaporizer(s) 10 may be of any type(s) described herein and/or may have any of the features, functions, structures, devices, modules and the like described herein. In particular, the hand-held personal vaporizer(s) 10 will include a communication module configured to communicate at least one way over a network 50. Such may include a receiver and/or a transponder configured for communication over the network. Such may include wireless technology and/or a wired technology, such as but not limited to a USB connector and/or a USB connection port disposed in/on the vaporizer. Accordingly, the vaporizer is able to communicate over the network, either sending or receiving messages (or both). The communication module may be in communication with a control module of the vaporizer which may track states of the device, control the operation thereof and/or manage accounts. Accordingly, such functions may be tracked, observed, supervised and/or controlled by the third-party control module 52 wherein communication is established between the third-party control module and the control module of the vaporizer itself. Such communication may serve to permit the third-party control module to extend the features, benefits, functions, memory, etc. of the control module and the third-party control module may operate subordinately to the control module of the vaporizer for one or more functions/purposes.

The control module of the vaporizer may include instructions and/or structure such that operation of the device may only occur if and/or when the third-party control module issues an authorization signal and that signal is received by the vaporizer. The vaporizer may include instructions wherein such a signal is only valid for a particular period of time and/or for a particular sized dosage, wherein the vaporizer automatically ceases function when such is expired and/or completed.

The illustrated network may be the internet, an intranet, a local wireless network, or the like or combinations thereof. The network permits communication between coupled systems/devices/modules.

The illustrated third-party control module includes instructions for observation, tracking, management, and/or control of the hand-held personal vaporizer over a network. The third-party control module includes a communication module similar to that of the hand-held personal vaporizer. Wherein the vaporizer only establishes one-way communication over the network, the third-party control module is at least able to communicate in a corresponding manner that matches with the vaporizer (i.e. at least receives of the vaporizer only transmits messages, at least transmits if the vaporizer only receives). The third-party control module also includes a user interface module configured to permit the user thereof to interact with the same and to observe states, historical data, characteristics, account settings, and the like and may also permit the user to issue commands, authorizations, change settings, connect with additional vaporizers and the like and combinations thereof. The user interface will generally include a display and a user input device (touch screen, mouse, keyboard, etc.).

Advantageously, the illustrated system of hand-held personal vaporizer(s) in communication with a third-party control module over a network allows for remote monitoring and/or control regarding status and/or usage of the connected vaporizers. Accordingly, such may be utilized by pharmacies, doctors, clinicians, therapists, guardians, and others in supervisory positions to be able to influence, monitor and/or otherwise control usage of vaporizers by those who are using the same. Further, even wherein the third-party control module is an application on one's own smartphone or tablet, the user may be able to monitor their own usage and/or may be reminded of their historical usage and/or goals in the act of authorizing the usage, since the display of the smart phone or tablet is much larger than that of the vaporizer. Accordingly, a great deal of flexibility, control, scripting, information display and the like may be incorporated into operation of the hand-held device without the added bulk that would come with it if such a display were incorporated into the vaporizer itself.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, although the illustrated hand-held personal vaporizer is of a particular shape (similar to a cigarette or e-cigarette), the various aesthetic designs that such a device may take, while still being hand-held and personal, are plethoric.

Additionally, although the figures illustrate hand-held personal vaporizers, it is envisioned that one or more embodiments of the systems described herein may include one or more vaporizers that are not hand-held and/or not personal.

It is also envisioned that there may be a plurality of third-party control modules that may be in communication with a single vaporizer.

Finally, it is envisioned that the components of the device may be constructed of a variety of materials, including but not limited to plastics, resins, rubbers, ceramics, metals, natural fibers, woods, composites and the like and combinations thereof.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A hand-held personal vaporizer, comprising:
a. a cylindrical housing having a mouthpiece and an elongated region opposite the mouthpiece;
b. a vaporizer disposed within the cylindrical housing;
c. a vaporizer control module functionally coupled to the vaporizer and configured to control function of the vaporizer;
d. a display on the cylindrical housing and functionally coupled to the vaporizer control module; and
e. a push button disposed on the elongated region of the cylindrical housing, functionally coupled to the vaporizer control module, and including a fingerprint scanner.

2. The vaporizer of claim 1, further comprising a locking module in communication with the vaporizer control module, functionally coupled to the fingerprint scanner, and configured to securely lock the vaporizer from use if an authorized fingerprint is not detected through the fingerprint scanner.

3. The vaporizer of claim 1, further comprising a preference module in communication with the vaporizer control module, configured to store preference settings for a stored user account.

4. The vaporizer of claim 1, further comprising a dosage settings module functionally coupled to the vaporizer control module and configured to store dosage settings per user.

5. The vaporizer of claim 1, further comprising a data storage module in communication with the vaporizer control module, configured to store user data, such as dosage and preferences from the vaporizer.

6. The vaporizer of claim 1, wherein a second fingerprint scanner functionally coupled to the vaporizer control module is located remote from the vaporizer on a personal computing device and wherein the vaporizer control module only allows operation of the vaporizer when authorized fingerprints are detected at both the first and second fingerprint scanners.

7. The vaporizer of claim 1, further comprising a communication module in communication with the vaporizer control module, configured to provide wireless communication to the modules and components of the vaporizer.

8. The vaporizer of claim 1, wherein the fingerprint scanner is disposed on a flat platform on a top surface of the cylindrical housing, under which there is a button mechanism.

9. The vaporizer of claim 1, wherein a remote locking module is functionally coupled to the vaporizer control module and is located remote from the vaporizer on a personal computing device and wherein the vaporizer control module only allows operation of the vaporizer when an authorized fingerprint is detected at fingerprint scanners and the remote locking module authorizes the use.

10. The vaporizer of claim 1, wherein the vaporizer control module automatically restricts use of the vaporizer after a predefined script is executed.

11. A hand-held personal vaporizer, comprising:
a. a cylindrical housing having a mouthpiece and an elongated region opposite the mouthpiece;
b. a vaporizer disposed within the cylindrical housing;
c. a vaporizer control module functionally coupled to the vaporizer and configured to control function of the vaporizer;
d. a display on the cylindrical housing and functionally coupled to the vaporizer control module;
e. a push button disposed on the elongated region of the cylindrical housing, functionally coupled to the vaporizer control module; and
f. a fingerprint scanner functionally coupled to the push button and configured to scan a fingerprint.

12. The vaporizer of claim 11, further comprising a locking module in communication with the vaporizer control module, functionally coupled to the fingerprint scanner, and configured to securely lock the vaporizer from use if an authorized fingerprint is not detected through the fingerprint scanner; wherein the vaporizer control module automatically restricts use of the vaporizer after a predefined script is executed.

13. The vaporizer of claim 12, further comprising a preference module in communication with the vaporizer control module, configured to store preference settings for a stored user account.

14. The vaporizer of claim 13, further comprising a dosage settings module functionally coupled to the vaporizer control module and configured to store dosage settings per user.

15. The vaporizer of claim 14, further comprising a data storage module in communication with the vaporizer control module, configured to store user data, such as dosage and preferences from the vaporizer.

16. The vaporizer of claim 15, wherein a second fingerprint scanner functionally coupled to the vaporizer control module is located remote from the vaporizer on a personal computing device and wherein the vaporizer control module only allows operation of the vaporizer when authorized fingerprints are detected at both the first and second fingerprint scanners.

17. The vaporizer of claim 16, further comprising a communication module in communication with the vaporizer control module, configured to provide wireless communication to the modules and components of the vaporizer.

18. The vaporizer of claim 17, wherein the fingerprint scanner is disposed on a flat platform on a top surface of the cylindrical housing, under which there is a button mechanism.

19. The vaporizer of claim 18, wherein a remote locking module is functionally coupled to the vaporizer control module and is located remote from the vaporizer on a personal computing device and wherein the vaporizer control module only allows operation of the vaporizer when an authorized fingerprint is detected at fingerprint scanners and the remote locking module authorizes the use.

20. A hand-held personal vaporizer, comprising:
a. a cylindrical housing having a mouthpiece and an elongated region opposite the mouthpiece;
b. a vaporizer disposed within the cylindrical housing;
c. a vaporizer control module functionally coupled to the vaporizer and configured to control function of the vaporizer;
d. a display on the cylindrical housing and functionally coupled to the vaporizer control module;
e. a push button disposed on the elongated region of the cylindrical housing, functionally coupled to the vaporizer control module;
f. a fingerprint scanner functionally coupled to the push button and configured to scan a fingerprint; wherein the fingerprint scanner is disposed on a flat platform on a top surface of the cylindrical housing, under which there is a button mechanism;
g. a locking module in communication with the vaporizer control module, functionally coupled to the fingerprint scanner, and configured to securely lock the vaporizer from use if an authorized fingerprint is not detected through the fingerprint scanner; wherein the vaporizer control module automatically restricts use of the vaporizer after a predefined script is executed;
h. a preference module in communication with the vaporizer control module, configured to store preference settings for a stored user account;
i. a dosage settings module functionally coupled to the vaporizer control module and configured to store dosage settings per user;
j. a data storage module in communication with the vaporizer control module, configured to store user data, such as dosage and preferences from the vaporizer;
k. a second fingerprint scanner functionally coupled to the vaporizer control module is located remote from the vaporizer on a personal computing device and wherein the vaporizer control module only allows operation of the vaporizer when authorized fingerprints are detected at both the first and second fingerprint scanners;
l. a communication module in communication with the vaporizer control module, configured to provide wireless communication to the modules and components of the vaporizer; and
m. a remote locking module is functionally coupled to the vaporizer control module and is located remote from the vaporizer on a personal computing device and wherein the vaporizer control module only allows operation of the vaporizer when an authorized fingerprint is detected at fingerprint scanners and the remote locking module authorizes the use.
